(19) United States
(12) Patent Application Publication
(10) Pub. No.: US 2025/0235283 A1
(43) Pub. Date: Jul. 24, 2025

(54) MEDICAL DEVICE PACKAGING CONFIGURABLE FOR DEVICE PREPARATION

(71) Applicant: EDWARDS LIFESCIENCES CORPORATION, Irvine, CA (US)

(72) Inventors: Robert Mac Carthy, Tustin, CA (US); Timothy William Hamilton, Orange, CA (US)

(73) Assignee: EDWARDS LIFESCIENCES CORPORATION, Irvine, CA (US)

(21) Appl. No.: 19/176,059

(22) Filed: Apr. 10, 2025

Related U.S. Application Data
(63) Continuation of application No. PCT/US2023/076932, filed on Oct. 15, 2023.
(60) Provisional application No. 63/379,434, filed on Oct. 13, 2022.

(51) Int. Cl. A61B 50/33; A61B 50/30

(52) U.S. Cl. CPC A61B 50/33; A61B 2050/3005

(57) ABSTRACT

Devices, systems, and/or methods discussed herein relate to medical device packaging that includes various features for packaging a medical device and/or preparing the medical device for use in a procedure. For example, packaging can include features to retain a medical device during distribution, such as foldable ends that are configured to cover the medical device. Further, the packaging can include features that are configurable to elevate one end of packaging to assist in preparing the medical device for a procedure, such as to flush the medical device in an elevated state. Moreover, the packaging can include features to hold and/or dispose of a fluid in an efficient manner.

### RELATED APPLICATION(S)

This application is a continuation of International Patent Application No. PCT/US2023/076932, filed Oct. 15, 2023, which claims the benefit of U.S. Provisional Patent Application No. 63/379,434, filed on Oct. 13, 2022, the complete disclosures of which are hereby incorporated by reference in their entireties.

### BACKGROUND

### Field

The present disclosure generally relates to the field of medical device packaging.

### Description of Related Art

Medical devices are often packaged in containers to protect the medical devices and/or maintain a sterilized state of the medical devices during manufacturing, distribution, use, or at other times. For example, a medical device can be packaged into a container to prevent the medical device from being contaminated by microorganisms (e.g., bacteria, virus, pathogens etc.) and/or from being damaged while being moved from one location to another.

### SUMMARY

Described herein are devices, methods, and/or systems for packaging medical devices in packaging that is configurable to contain/protect the medical devices during distribution, shipment, or at other times, to orient the medical devices in a particular manner to assist in preparing the medical devices for use in a procedure, and/or to facilitate other functions.

For purposes of summarizing the disclosure, certain aspects, advantages, and/or features are described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIG. 1-1 illustrates an exploded side-view of packaging that includes one or more of the features discussed herein in accordance with one or more examples.

FIG. 1-2 illustrates a side view of the packaging from FIG. 1-1 with the medical device disposed within the tray and the retainer coupled thereto to secure the medical device in accordance with one or more examples.

FIG. 1-3 illustrates a top view of the packaging from FIG. 1-2 with the medical device disposed within the tray and the retainer removed in accordance with one or more examples.

FIG. 2A illustrates a perspective view of the tray from FIG. 1-1 in accordance with one or more examples,

FIG. 2B illustrates a top view of the tray from FIG. 1-1 in accordance with one or more examples.

FIG. 2C illustrates a side view of the tray from FIG. 1-1 in accordance with one or more examples.

FIG. 3A illustrates a perspective view of the retainer from FIG. 1-1 in accordance with one or more examples.

FIG. 3B illustrates a top view of the retainer from FIG. 1-1 in accordance with one or more examples.

FIG. 3C illustrates a side view of the retainer from FIG. 1-1 in accordance with one or more examples.

FIG. 4 illustrates an example configuration of a tray in a device-preparation state, wherein an end portion of the tray is folded/flexed/bent towards a bottom side of the tray in accordance with one or more examples.

FIG. 5 illustrates an example configuration of a tray in a device-preparation state, wherein an end portion of the tray is folded/flexed into multiple sections towards a bottom side of the tray in accordance with one or more examples.

FIG. 6 illustrates an example configuration of packaging in a device-preparation state, wherein an end portion of a tray is folded/flexed towards a bottom side of the tray and received/held by a retainer in accordance with one or more examples.

FIG. 7 illustrates an example configuration of packaging in a device-preparation state, wherein a tray rests on or couples to a retainer at multiple locations to elevate one end of the tray in accordance with one or more examples.

FIG. 8 illustrates an example configuration of packaging in a device-preparation state, wherein a tray rests on or couples to a retainer at a location to elevate one end of the tray in accordance with one or more examples.

FIG. 9 illustrates an example configuration of packaging in a device-preparation state, wherein multiple portions of a retainer are coupled to or otherwise placed adjacent to each other along a bottom side of the retainer to elevate one end of a tray in accordance with one or more examples.

FIG. 10 illustrates an example configuration of packaging in a device-preparation state, wherein multiple portions of a retainer are coupled to or otherwise placed adjacent to each other along a top side of the retainer to elevate one end of a tray in accordance with one or more examples.

FIG. 11 illustrates an example device-preparation procedure/operation, wherein packaging is configured in a device-preparation state/configuration and a medical device is flushed in an upward manner in accordance with one or more examples.

FIG. 12 illustrates an example device-preparation procedure/operation, wherein packaging is configured in a device-preparation state/configuration and a medical device is flushed in a downward manner in accordance with one or more examples.

FIG. 13A illustrates a perspective view of packaging with one or more end portions that are configurable to cover/retain a medical instrument in accordance with one or more examples.

FIG. 13B illustrates a top view of the packaging from FIG. 13A in accordance with one or more examples.

FIG. 13C illustrates a side view of the packaging from FIG. 13A in accordance with one or more examples.

FIG. 14A illustrates a side view of the packaging from FIG. 13A in a configuration with end portions folded to cover a medical instrument in accordance with one or more examples.

FIG. 14B illustrates a top view of the packaging from FIG. 14A in accordance with one or more examples.

FIG. 15 illustrates a side view of the packaging from FIG. 13A in a configuration with an end portion folded back to elevate/raise one end of the packaging in accordance with one or more examples.

FIG. 16 illustrates example packaging that includes one or more plugs/stoppers/caps/scaling members that are configured to seal one or more holes in a fluid retention feature in accordance with one or more examples.

FIG. 17-1 illustrates the example packaging from FIG. 16 with the plug located at a position a distance from a hole in which the plug will be implemented in accordance with one or more examples.

FIG. 17-2 illustrates the examples packaging from FIG. 16 with the plug removed from the position in accordance with one or more examples.

FIG. 17-3 illustrates the examples packaging from FIG. 16 with the plug placed in the hole in accordance with one or more examples.

FIG. 18 illustrates the example packaging from FIG. 16 with the plug removed and fluid draining from the packaging in accordance with one or more examples.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for ease of description to describe the positional relationship between element(s)/structures(s), as illustrated in the drawings. Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

Medical instruments are often prepared for a procedure by flushing the instruments with a fluid, such as saline or another liquid. This can remove air from the instruments and/or otherwise prepare the instruments for a procedure. In some cases, a user holds the medical instrument at an angle to allow for upward or downward flushing of the medical instrument, thereby allowing air to escape from the medical instrument. For example, a user may hold a catheter with one hand in an elevated manner and hold a syringe or another device in the other hand to flush a fluid through the catheter. However, it can be difficult for the user to hold and flush a medical instrument, particularly when the medical instrument is relatively large. Further, in some cases, the user can flush a fluid through the medical instrument or otherwise treat the instrument with the fluid while the instrument is in the packaging, which can cause the packaging to fill with the fluid. However, the size, shape, and/or flexibility of the packaging and/or the medical instrument can make it difficult to dispose of the fluid from the packaging, such as to pour the liquid from the packaging without spilling.

The present disclosure relates to, among other things, devices, methods, and/or systems for packaging that includes various features configurable for preparing the medical instruments for use in a medical procedure. For example, packaging can include features to retain a medical instrument during distribution and features that are configurable to elevate one end of packaging and flush the medical instrument with a fluid during preparation of the medical instrument for a procedure. This can assist the user in flushing the medical instrument in a more efficient manner, while minimizing the reliance on other equipment. Further, the packaging can include features to hold and/or dispose of a fluid in an efficient manner, features to retain the medical instrument in the packaging with reliance on less packaging (in comparison to other solutions), and/or a variety of other features.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

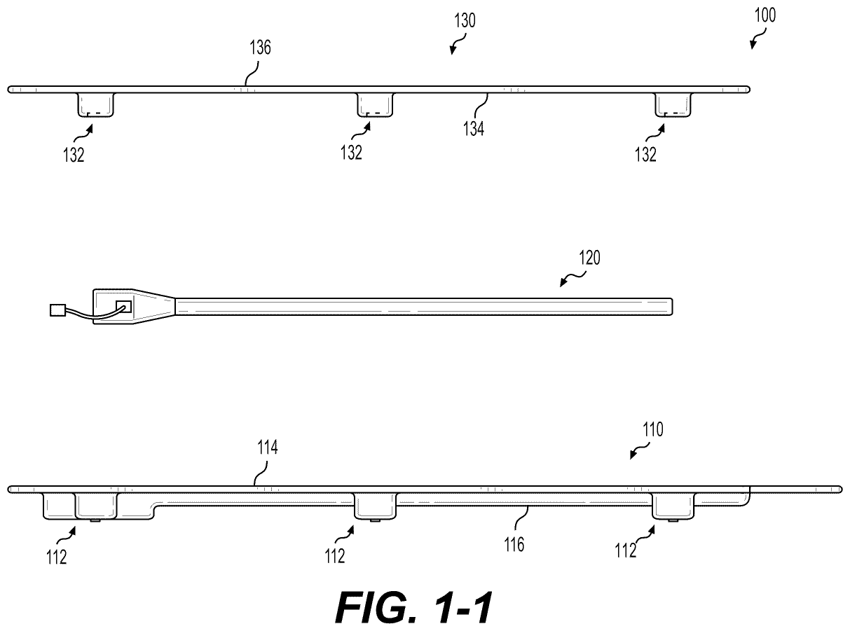

FIGS. 1-1 through 1-3 illustrate medical device packaging 100 configurable to retain a medical device/instrument and/or orient/position the medical device for preparation of the medical device for a procedure in accordance with one or more examples. The medical device packaging 100 (also referred to as “the medical device system/kit 100”) can include a tray/base(s) 110 (e.g., first packaging component/piece) configured to hold/retain/receive/couple to a medical device/instrument(s) 120 and/or a retainer/cover/lid(s) 130 (e.g., second packaging component/piece) configured to removably couple/attach to or otherwise engage with the tray 110 in a variety of manners to retain/hold the medical device 120 within the tray 110, assist in preparing the medical device 120 for a procedure, etc.

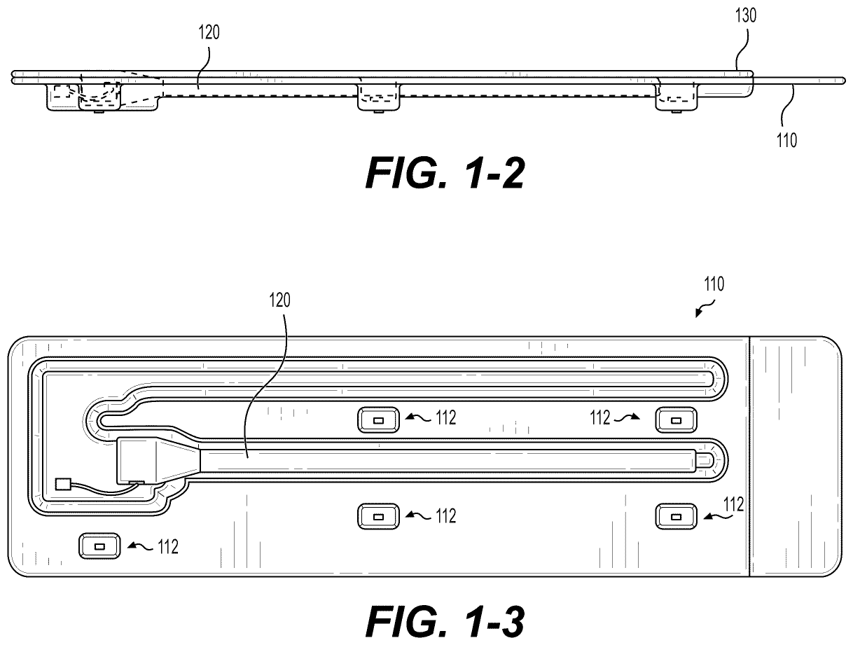

FIG. 1-1 illustrates an exploded side-view of the packaging 100 (e.g., with the components separated from each other). FIG. 1-2 illustrates a side view of the packaging 100 with the medical device 120 disposed within the tray 110 and the retainer 130 coupled thereto to secure the medical device 120 within the tray 110. FIG. 1-3 illustrates a top view of the packaging 100 with the medical device 120 disposed within the tray 110 and the retainer 130 removed. Although the packaging 100 is illustrated with several components/elements (e.g., packaging pieces), the packaging 100 can include one or more components/elements. For example, the packaging 100 can refer to the tray 110, the retainer 130, and/or other components/elements that may or may not be presented in the figures. The packaging 100 can be dimensioned to the size/shape/form/dimension of the medical device 120. Although various figures illustrate a single medical device, the packaging 100 can be configured to receive/retain any number of medical devices and/or other types of devices.

The tray 110 and/or the retainer 130 (and/or the medical instrument 120, in some cases) can include any number of engagement/mating/coupling/attachment features configured to couple the components to each other. In the example of FIG. 1-1 through 1-3 and elsewhere, the tray 110 includes one or more engagement features 112 configured to couple/receive/engage with one or more engagement features 132 of the retainer 130. Although the one or more engagement features 112/132 are illustrated in particular forms, the one or more engagement features 112/132 can be implemented as another type of engagement/mating feature and/or the illustrated/discussed types of engagement features can be swapped. In some implementations, the one or more engagement/coupling/mating features 112, 132 (and/or other engagement/coupling/mating features discussed herein) can include features that generally include similarly shaped, yet opposing features configured to implement a friction/pressure type of coupling, such as a snap type coupling.

The tray 110 and/or the retainer 130 can include one or more retention features configured to couple to or otherwise receive the medical device 120 and/or hold the medical device 120 in place. For example, the tray 110 and/or the retainer 130 can include one or more contours/recessed portions/projections that are generally designed/formed to the shape/form of the medical instrument 120. The shape/dimensions of a contour(s) can be adapted/configured to match the shape/dimensions of the associated medical device 120. In various examples discussed herein, the tray 110 is illustrated with a recessed portion configured to receive/hold the medical device 120; however, the tray 110 can include other types of retention features to hold the medical instrument 120.

The tray 110 and/or the retainer 130 can be implemented as fully/substantially/semi rigid or flexible components. The tray 110 and/or the retainer 130 (and/or other packaging components of the packaging 100) can include plastic, paper, metal, composite materials, or other materials. In some instances, the tray 110 and/or the retainer 130 (and/or other packaging components of the packaging 100) are made from sustainable/recyclable/compostable materials, while in other instances the tray 110 and/or the retainer 130 (and/or other packaging components of the packaging 100) are made from non-sustainable/non-recyclable/non-compostable materials.

The packaging 100 can be configured to implement one or more configurations/states to facilitate various functionality. For example, the packaging 100 can be configured in a packaged configuration/state, where the medical device 120 is substantially/fully retained/secured within the packaging 100, which can be implemented during shipment, distribution, storage, and/or at other times. Here, the packaging 100 holds the medical device 120 in place and/or provides protection for the medical device 120. To illustrate, in an example packaged configuration, such as that shown in FIG. 1-2, the one or more engagement features 132 of the retainer 130 engage/couple to the one or more engagement features 112 of the tray 110, such that a bottom side 134 of the retainer 130 is adjacent to or contacts a top side 114 of the tray 110 (e.g., the retainer 130 is configured in a first/packaged orientation).

Further, the packaging 100 can be configured in a device-preparation configuration/state, where at least a portion of the packaging 100 is propped up/elevated at one end and/or otherwise oriented/positioned to prepare/treat the medical device 120 for a procedure. For instance, the tray 110 can be reconfigured and/or coupled to the retainer 130 in another manner to raise/elevated one end of the tray 110 when the tray 110 is positioned on a substantially planar/flat surface, resulting in one end of the medical instrument 120 being elevated/raised relative to the other end of the medical instrument 120. To illustrate, in an example device-preparation configuration, the bottom side 134 or top side 136 of the retainer 130 is positioned adjacent to or coupled to a bottom side 116 of the tray 110 and/or one end of the tray 110 is folded to raise/lift/tilt one end of the tray 110 relative to the other end of the tray 110. In some implementations, the medical device 120 is elevated to assist in flushing the medical device 120 with a fluid, such as saline, wherein the elevated orientation/configuration can minimize/eliminate air bubbles and/or particulates in the medical device 120. For example, the medical device 120 can include a tube, wherein saline or another fluid is forced into one side of the tube while air/particulates exit/escape from the other side of the tube.

As such, the packaging 100 is configurable for various functions/purposes. For example, the packaging 100 can be configured at certain times for distribution/shipment of the medical device 120 and reconfigured thereafter or at other times to assist in preparing the medical device 120 for a procedure.

FIGS. 2A-2C illustrate example details of the tray 110 in accordance with one or more examples. In particular, FIG. 2A illustrates a perspective view of the tray 110, FIG. 2B illustrates a top view of the tray 110, and FIG. 2C illustrates a side view of the tray 110. Although various example features are shown in particular configurations for the tray 110, other features can be implemented and/or the illustrated features can be configured in different manners.

As shown, the tray 110 includes a retention section 202 configured to retain/hold a medical instrument(s) (not shown). In particular, the retention section 202 includes a retention feature 204 that is configured to receive/hold a medical instrument in a firm or loose manner within the tray 110. For example, the retention feature 204 can include a recessed portion that extends along a length of the tray 110, as shown, which can be configured to hold a shaft/elongate member of a medical instrument in a fixed position within the tray 110 (e.g., the medical instrument is fully secured) or in a more loosely defined position within the tray 110 (e.g., the medical instrument is more loosely secured, such that the medical instrument can move somewhat within the recessed portion). Although the retention feature 204 is illustrated as a recessed portion, the retention feature 204 can be implemented in any number of ways, such as using one or more projections or other features to receive/hold one or more medical instruments. In the example shown, multiple retention features are implemented, wherein a first retention feature 204(A) is configured to hold/receive a first medical instrument and a retention feature 204(B) is configured to receive hold a second medical instrument. The retention features 204(A) and 204(B) may or may not be joined, although they are joined in this example. In some cases, the retention feature 204(A)/204(B) can be used as a reservoir to collect/contain a fluid, such as when a medical device is flushed with a fluid while being disposed within the tray 110. The retention features 204(A)/204(B) can be a continuous portion or separate sections.

In some implementations, the tray 110 includes an end portion/section 206 (also referred to as “the foldable/bendable portion 206”) that is adjacent to and/or coupled to the retention section 202, wherein the end portion 206 can flex/fold/bend relative to the retention section 202. For instance, the end portion 206 can be implemented in a relatively straight orientation, such as that shown in FIGS. 2A-2C, which can be implemented during a packaged configuration. Further, in a device-preparation configuration, the end portion 206 can fold down towards the bottom side 116 of the tray 110 to elevate that end of the tray 110. In some cases, the end portion 206 is folded/flexed relative to the retention section 202 along a line 208, which can include a scored portion (e.g., a formed groove in the material), a relatively thin piece of material (e.g., with a thickness less than a threshold or another portion of the tray 110), a hinge, or another feature that assists in providing a hinge point. The end portion 206 can generally be dimensioned such that flexing the end portion 206 towards the bottom side 116 of the tray 110 can cause the end portion 206 to extend below the bottom-most surface of the tray 110 and elevate/raise that end of the tray 110. For example, the end portion 206 can be longer than a depth of the tray 110, wherein the depth of the tray 110 is illustrated in the side view of FIG. 2C.

The tray 110 can include an elongate and/or upper structure/element 210 and the one or more engagement/coupling features 112 disposed thereon. However, the tray 110 can take other forms. The one or more engagement features 112 can be configured to couple/attached to the retainer 130 (not shown) in a variety of manners to hold a medical device and/or to elevate one or more portions of the tray 110. Further, in some cases, the one or more engagement features 112 can be configured to couple to each other, which can assist in elevating the tray 110 and/or providing other functionality. In this example, multiple engagement features 112(A)-(C) are implemented, with each engagement feature 112 aligned with a corresponding engagement feature of the retainer 130. The engagement features 112 are implemented as recessed portions off the top side/surface 114/upper structure 210 and projections/protrusions off the bottom side 116; however, the engagement features 112 can be implemented in other manners. The bottom side 116 is an opposing side to the top side 114.

In some implementations, the engagement feature 112(A)(1) is merely intended to engage/couple to an engagement feature of the retainer 130 in a device-preparation configuration. However, the engagement feature 112(A)(1) can be configured to engage with an engagement feature of the retainer 130 in a packaged configuration, in some cases. Further, in some implementations, the engagement feature 112(A)(2) is merely intended to engage/couple to an engagement feature of the retainer 130 in a packaged configuration. However, the engagement feature 112(A)(2) can be configured to engage with an engagement feature of the retainer 130 in a device-preparation configuration, in some cases.

The engagement feature 112(B)(2) is representative of any of the engagement features 112(A)-112(C). The engagement feature 112(B)(2) can be implemented in a variety of forms/manners to couple to another component/element and facilitate a packaged configuration and/or a device-preparation configuration. For instance, the engagement feature 112(B)(2) can include one or more protrusions, detents, indentations, recesses, cavities, depressions, holes, projections, snap-fit features, pins, hooks, flanges, edges, nipples, fasteners, adhesives, etc. A few non-limiting example features/forms (e.g., end portions for the engagement feature 112(B)(2)) that can be implemented for the engagement feature 112(B)(2) are shown in the callout of FIG. 2C.

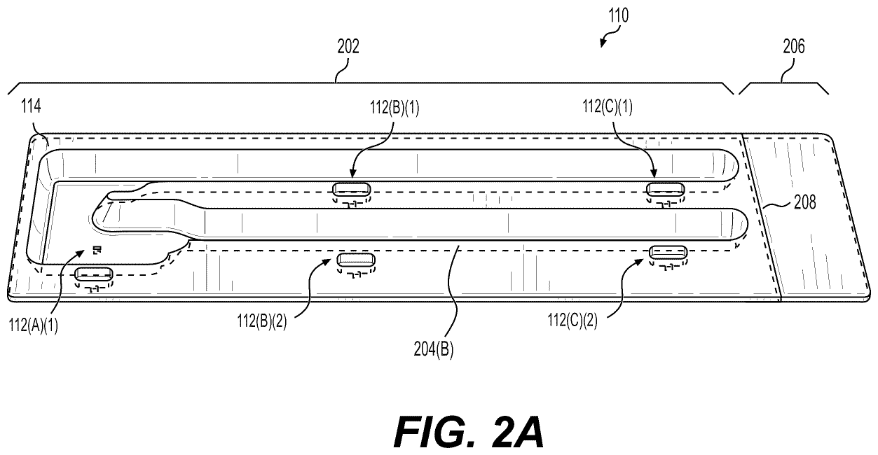

FIGS. 3A-3C illustrate example details of the retainer 130 in accordance with one or more examples. In particular, FIG. 3A illustrates a perspective view of the retainer 130, FIG. 3B illustrates a top view of the retainer 130, and FIG. 3C illustrates a side view of the retainer 130. Although various example features are shown in particular configurations for the retainer 130, other features can be implemented and/or the illustrated features can be configured in different manners. Further, although various features are discussed in the context of the retainer 130 (and/or the tray 110), the features can be implemented on other packaging elements, such as a removable insert or another packaging component that may not retain/hold a medical instrument.

The retainer 130 can include an elongate and/or upper structure/element 302 and the one or more engagement/coupling features 132 disposed thereon. However, the retainer 130 can take other forms. The one or more engagement features 132 can be removably attachable/couplable to the tray 110 (not shown), such as in a variety of manners to hold a medical device in the tray 110 and/or to elevate one or more portions of the tray 110. Further, in some cases, the one or more engagement features 132 can be configured to couple to each other, such as when the retainer 130 is bendable or separatable into multiple pieces. In this example, multiple engagement features 132(A)-(C) are implemented, with each engagement feature 132 aligned with a corresponding engagement feature of the tray 110. The engagement features 132 are implemented as recessed portions off the top side/surface 136/upper structure 302 and projections/protrusions off the bottom side/surface 134; however, the engagement features 132 can be implemented in other manners. Although a particular number of engagement features 132 at specific locations are shown, the retainer 130 can include any number of engagement features positioned at a variety of locations on the retainer 130.

The engagement feature 132(A) is representative of any of the engagement features 132(A)-132(C). The engagement feature 132(A) can be implemented in a variety of forms/manners to couple to another component/element and facilitate a packaged configuration and/or a device-preparation configuration. For instance, the engagement feature 132(A) can include one or more protrusions, detents, indentations, recesses, cavities, depressions, holes, projections, snap-fit projections, pins, hooks, flanges, nipples, fasteners, adhesives, etc. A few non-limiting example features/forms (e.g., end portions for the engagement feature 132(A)) that can be implemented for the engagement feature 132(A) are shown in the callout of FIG. 3C.

In some implementations, in a packaged configuration, the engagement feature 132(A) of the retainer 130 is configured to couple to the engagement feature 112(A)(2) of the tray 110, the engagement feature 132(B)(1) is configured to couple to the engagement feature 112(B)(1), the engagement feature 132(B)(2) is configured to couple to the engagement feature 112(B)(2), the engagement feature 132(C)(1) is configured to couple to the engagement feature 112(C)(1), and/or the engagement feature 132(C)(2) is configured to couple to the engagement feature 112(C)(2). In such configuration/state, the retainer 130 can be attached to the tray 110 to secure a medical instrument within the tray 110

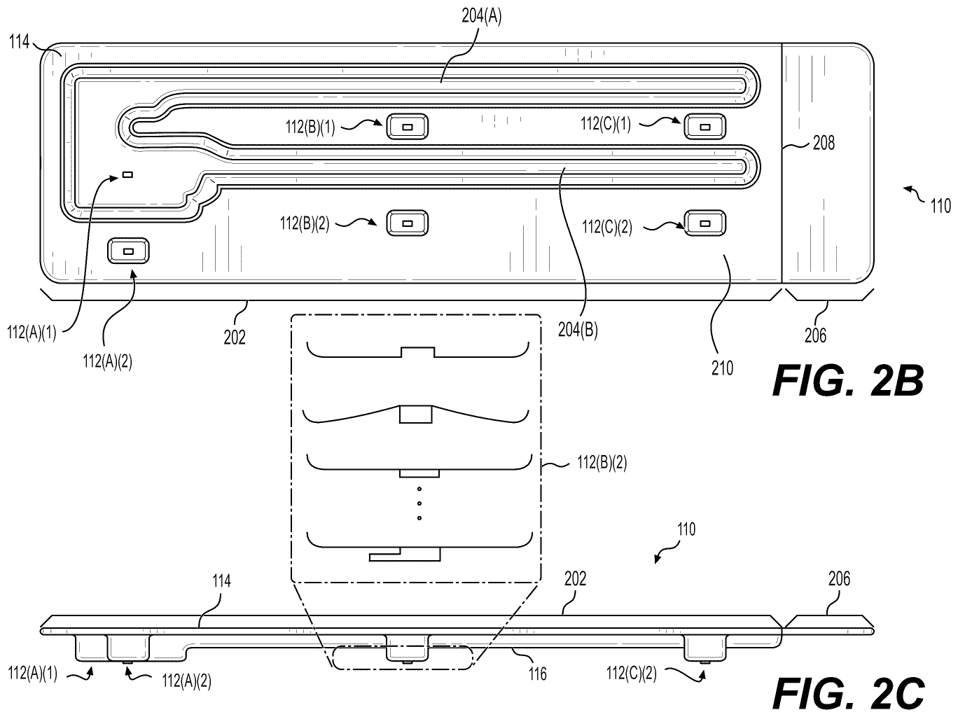

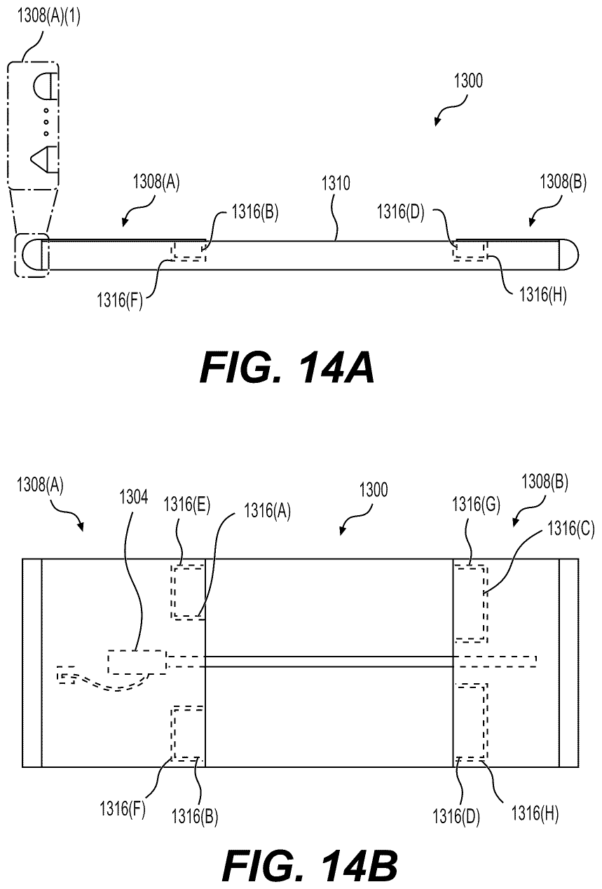

FIG. 4 illustrates an example configuration of the tray 110 in a device-preparation state, wherein the end portion 206 is folded/flexed/bent towards the bottom side 116 of the tray 110 in accordance with one or more examples. Here, the end portion 206 is folded/flexed/bent towards the bottom side 116 to provide a support stand/structure for one end of the tray 110. This causes one end of the tray 110 and one end of the medical device 120 disposed therein to be raised higher than the other end of the tray 110/medical device 120. For example, the end portion 206 is folded/flexed/bent downwards from an axis/plane 402 (e.g., longitudinal axis/plane), such that the end portion 206 extends past an axis/plane 404 that is aligned to a bottom most surface/component of the tray 110. As such, a distance between the top surface 114 and a bottommost/lowest element of the tray 110 at one end (i.e., the right-hand side of FIG. 4) is greater than a distance between the top surface 114 and a bottom most/lowest element of the tray 110 at the other end (i.e., the left-hand side of FIG. 4). In contrast, in a packaged configuration, the end portion 206 can generally be straight relative to the axis/plane 402 (e.g., aligned with the axis/plane 402; also referred to as “the longitudinal axis 402”) (as shown in FIG. 2C, for example) or bent/flexed/folded towards the top side 114 of the tray 110 (as shown in FIGS. 14A and 14B, for example).

In FIG. 4, the end portion 206 of the tray 110 is oriented in a substantially perpendicular manner relative to a surface 406 on which the tray 110 is positioned (and/or relative to the rest of the tray 110, such as the retention section 202), However, the end portion 206 can be oriented in other manners (e.g., by different degrees) and still provide support to raise one end of the tray 110. In some implementations, the end portion 206 is configured to fold/bend/flex towards the bottom side 116 in a relatively loose manner until a particular orientation is reached (e.g., the end portion 206 can lock into place).

In implementations, the terms bent, flexed, folded, and/or variations thereof refer to instances in which a component/element includes or does not include a crease. In other words, such terms are broadly used to refer to implementations where an element is bent/flexed/folded with a crease and implementations where an element is bent/flexed/folded without a crease. Thus, although a crease may be shown in various figures, the crease may or may not be implemented/exist.

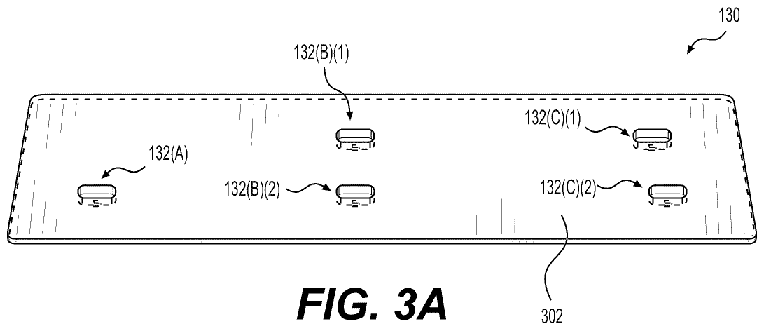

FIG. 5 illustrates an example configuration of the tray 110 in a device-preparation state, wherein the end portion 206 is folded/flexed into multiple sections towards the bottom side 116 of the tray 110 in accordance with one or more examples. As similarly discussed above with reference to FIG. 4, the end portion 206 is folded/flexed/bent towards the bottom side 116 to provide a support stand for one end of the tray 110. Here, the end portion 206 can bend at multiple locations/lines/features to form two sections 206(A) and 206(B) that incrementally flex/bend towards the bottom side 116 of the tray 110 (e.g., fold towards each other), as shown. Here, the section 206(B) contacts the bottom side 116 of the tray 110 to prop-up that side of the tray 110 on the surface 402. The section 206(B) can be held at least partly in place by the retention feature 204 (and/or the engagement feature 112(C)(2)). Although two folded portions/sections are illustrated, any number of portions/sections can be implemented. In some cases, a folded section (e.g., a distal most section) folds back towards and/or contacts the initial section that started the fold and/or another section. In some implementations, the end portion 206 is folded/flexed at multiple locations with the assistance of scoring (e.g., a cut groove), a relatively thin piece of material, a hinge, or another feature that assists in providing a hinge point.

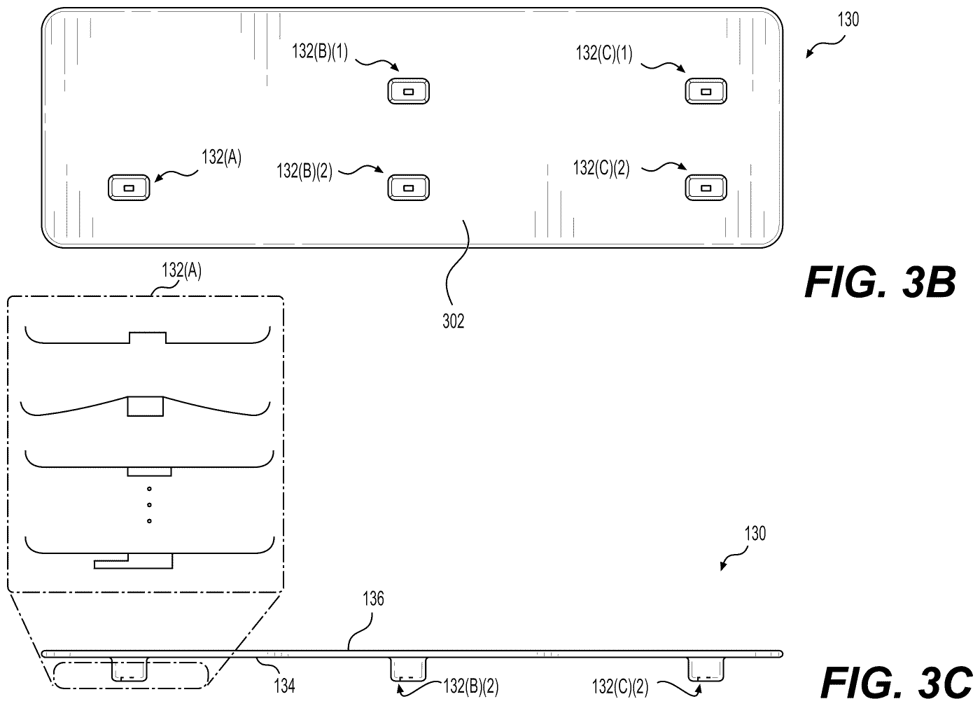

FIG. 6 illustrates an example configuration of the packaging 100 in a device-preparation state, wherein the end portion 206 of the tray 110 is folded/flexed towards the bottom side 116 of the tray 110 and received/held by the retainer 130 in accordance with one or more examples. Here, the retainer 130 is oriented up-side-down (relative to a packaged configuration) and positioned on the surface 406 with at least a portion of the retainer 130 positioned adjacent to the bottom side 116 of the tray 110 (e.g., underneath the tray 110). However, the retainer 130 can be positioned right-side-up and/or in other manners. The retainer 130 (e.g., an engagement features 13) can include a receiving/coupling feature 602 to receive and/or couple to the end portion 206. The coupling feature 602 can include a slot, recess, or other feature to receive/couple to the end portion 206. The coupling feature 602 can assist in maintaining the orientation of the end portion 206, such as in the perpendicular orientation shown. In some implementations, the engagement feature 112(C) of the tray 110 rests or couples to the engagement feature 132(A) of the retainer 130, as shown in the example of FIG. 6.

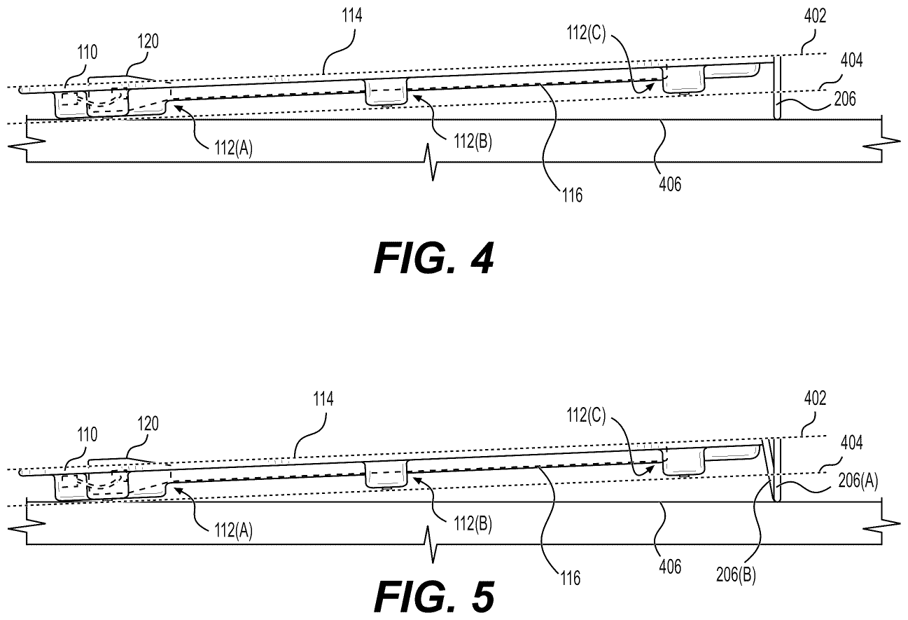

FIG. 7 illustrates an example configuration of the packaging 100 in a device-preparation state, wherein the tray 110 rests on or couples to the retainer 130 at multiple locations to elevate one end of the tray 110 in accordance with one or more examples. Here, the retainer 130 is oriented up-side-down (relative to a packaged configuration) and positioned on the surface 406 with at least a portion of the retainer 130 positioned adjacent to the bottom side 116 of the tray 110 (e.g., underneath the tray 110). However, the retainer 130 can be positioned right-side-up and/or in other manners. The retainer 130 can be positioned with either end of the retainer 130 positioned below the tray 110. As illustrated, the engagement feature 112(B) of the tray 110 (which can be representative of the engagement feature 112(B)(1) and/or 112(B)(2)) rests on or couples to the engagement feature 132(A)/132(C) of the retainer 130 (which can be representative of the engagement feature 132(A) when that end of the retainer 130 is positioned below the tray 110 or representative of the engagement feature 132(C)(1) and/or 132(C)(2) when the other end of the retainer 130 is positioned below the tray 110). Further, the engagement feature 112(C) of the tray 110 (which can be representative of the engagement feature 112(C)(1) and/or 112(C)(2)) rests on or couples to the engagement feature 132(B) of the retainer 130 (which can be representative of the engagement feature 132(B)(1) and/or 132(B)(2)). In examples, the same engagement feature of the retainer 130 is configured to couple to both a first side of the tray 110 (e.g., top side 114) when the retainer 130 is configured in a first orientation and a second side of the tray 110 (e.g., the bottom side 116) when the retainer is configured in a second orientation. In the example shown, the engagement feature 132(A)/132(C) is shorter (e.g., smaller depth dimension) than the engagement feature 132(B).

For ease of illustration in various figures, the engagement features 112 of the tray 110 are often illustrated as male features, while the engagement features 132 of the retainer 130 are often illustrated as female features. However, the features can be reversed or implemented in other ways.

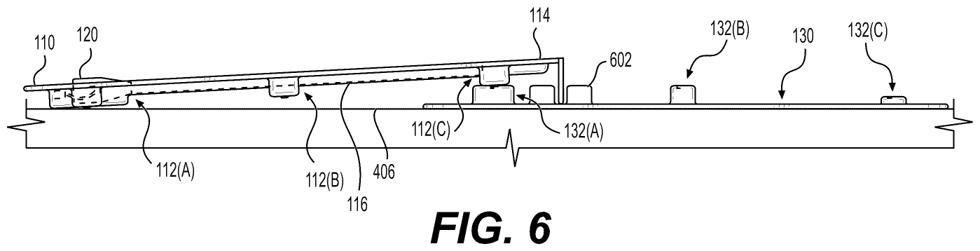

FIG. 8 illustrates an example configuration of the packaging 100 in a device-preparation state, wherein the tray 110 rests on or couples to the retainer 130 at a location to elevate one end of the tray 110 in accordance with one or more examples. Here, the retainer 130 is oriented up-side-down (relative to a packaged configuration) and positioned on the surface 406 with at least a portion of the retainer 130 positioned adjacent to the bottom side 116 of the tray 110 (e.g., underneath the tray 110). However, the retainer 130 can be positioned right-side-up and/or in other manners. The retainer 130 can be positioned with either end of the retainer 130 positioned below the tray 110. As illustrated, the engagement feature 112(C) of the tray 110 (which can be representative of the engagement feature 112(C)(1) and/or 112(C)(2)) rests on or couples to the engagement feature 132(A)/132(C) of the retainer 130 (which can be representative of the engagement feature 132(A) when that end of the retainer 130 is positioned below the tray 110 or representative of the engagement feature 132(C)(1) and/or 132(C)(2) when the other end of the retainer 130 is positioned below the tray 110).

Although the examples of FIGS. 7 and 8 illustrate the engagement features 112 and 132 coupled to each other to prop-up the tray 110, the tray 110 can rest on or couple to the retainer 130 without the engagement features 112 and 132 coupled to each other. For example, the tray 110 can be positioned relative to the retainer 130 such that an engagement feature 132 of the retainer 130 contacts a bottom portion of the tray 110 that is not an engagement feature 112.

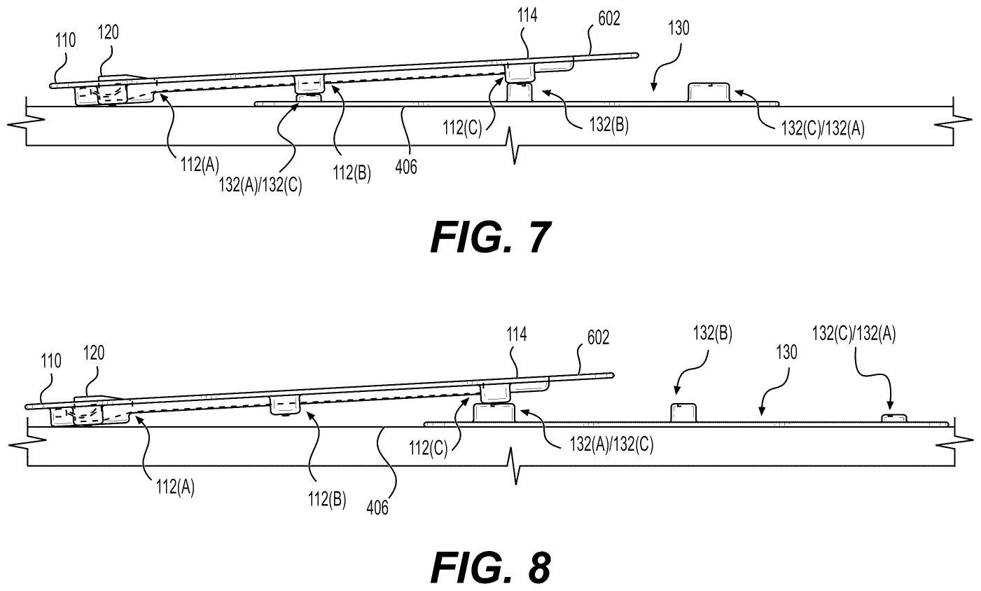

FIG. 9 illustrates an example configuration of the packaging 100 in a device-preparation state, wherein multiple portions 130(A) and 130(B) of the retainer 130 are coupled to or otherwise placed adjacent to each other along the bottom side 134 of the retainer 130 to elevate one end of the tray 110 in accordance with one or more examples. In examples, the engagement feature 132(A) rests on or couples to the engagement feature 132(C)(which can be representative of the engagement feature 132(C)(1) or 132(C)(2)). The portions 130(A) and 130(B) can be configured to provide an inclined surface for the tray 110 to rest on or couple to. In some cases, the engagement feature 132(A)/132(C) includes a modified structure and/or position on the retainer 130 to receive or couple to the engagement feature 132(C)/132(A). For example, the engagement feature 132(A) can be larger/longer than the engagement feature 132(C) and include a recessed portion that has a shape/form matching/similar to a shape/form of the engagement feature 132(C), such that the engagement feature 132(A) can receive the engagement feature 132(C). In some implementations, the engagement feature 112(B) is configured to couple to the engagement feature 132(B), such as through a hole/opening in the main/base structure/surface of the retainer 130 (which can allow the engagement features 112(B) and 132(B) to contact each other).

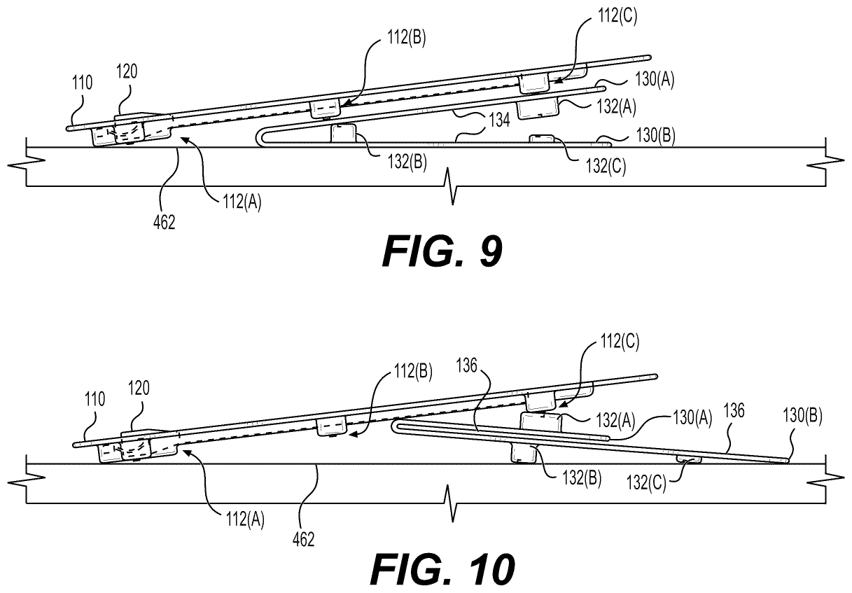

FIG. 10 illustrates an example configuration of the packaging 100 in a device-preparation state, wherein multiple portions 130(A) and 130(B) of the retainer 130 are coupled to or otherwise placed adjacent to each other along the top side 136 of the retainer 130 to elevate one end of the tray 110 in accordance with one or more examples. Here, the engagement feature 112(C) of the tray 110 can rest on or couple to the engagement feature 132(A) of the retainer 130, as shown. Although shown as being oriented with the left side of the retainer 130 oriented higher than the right side, the retainer 130 can be flipped such that the right side is higher than the left side (e.g., the retainer 130 is flipped).

In the examples of FIGS. 9 and 10, the retainer 130 includes two portions/sections 130(A) and 130(B) that can be integral or separate elements. In some implementations of an integral component, the multiple portions 130(A) and 103(B) can be flexed/bent/folded onto each other. Further, in some implementation, the retainer 130 is initially configured as an integral component (e.g., a single piece/component), wherein the retainer 130 is configured to be separated into multiple pieces (e.g., separable components). For example, the retainer 130 can include a scored portion (e.g., a formed groove in the material), a relatively thin piece of material, and/or other feature that facilitates separation of the retainer 130 into multiple pieces in an easy manner. The separate components can be coupled to or placed adjacent to each other in the manner shown in FIGS. 9 and/or 10.

Although two portions 130(A) and 130(B) are illustrated in FIGS. 9 and 10, the retainer 130 can be separated/sectioned into any number of portions. Further, although the multiple portions 130(A) and 130(B) are separated/defined at a particular location (e.g., between the engagement features 132(A) and 132(B)), the retainer 130 can be separated/defined at other locations. In examples, the configurations of FIGS. 9 and/or 10 can facilitate an increased incline, in comparison to other configurations.

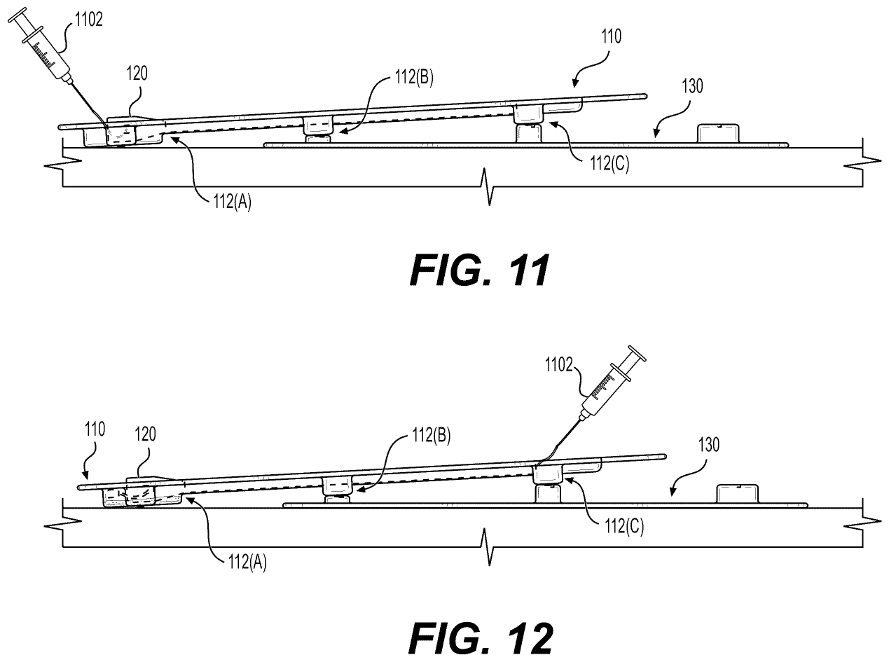

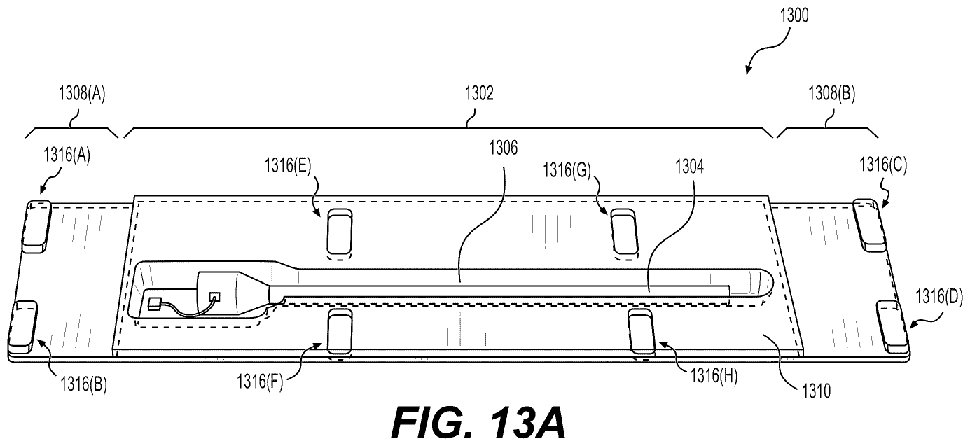

FIGS. 11 and 12 illustrate an example device-preparation procedure/operation, wherein the packaging 100 is configured in a device-preparation state/configuration to prepare the medical device 120 for a procedure in accordance with one or more examples. In various instances, the medical device/instrument 120 is prepared for a procedure by flushing/forcing a fluid into or through the medical device 120. For example, saline or another fluid can be injected into a tube, elongate member, or another cavity of the medical device 120 to prepare the medical device 120 with the saline/fluid and/or remove air/particulates from the medical device 120 (e.g., improve air egress). One or more of the device-preparation configurations discussed herein can be implemented to elevate one end of the medical device 120 to flush the medical device 120 with a fluid while preventing/minimizing/removing air that may otherwise collect in the medical device 120. Although various packaging configurations are discussed in the context of performing device-preparation procedures that includes flushing the medical device 120 with a fluid, other device-preparation procedures can be implemented with the packaging 100. Furthermore, the packaging 100 can be configured for other purposes. For ease of illustration, the examples of FIGS. 11 and 12 are discussed in the context of one end of the tray 110 being elevated/angled by a coupling to the retainer 130, such as in the configuration shown in FIG. 7. However, the tray 110 can be elevated in other manners, such as any of the configurations discussed herein.

As shown in FIGS. 11 and 12, a syringe 1102 or another device can be fluidly coupled to the medical device 120 (e.g., via a tube and/or port for the medical device 120), wherein the syringe 1102 can inject a fluid into the medical device 120. In an upward flush, such as that shown in FIG. 11, the fluid can travel from the left-hand side of the medical device 120 and exit out the right-hand side of the medical device 120. As the fluid travels up the medical device 120, air/particulates can also travel out the right-hand side of the medical device 120. In some cases, the fluid can pool/collect within the retention feature 204 and/or an engagement feature, such as the engagement feature 112(A). The tray 110, such as the engagement feature 112(A), can fill with the fluid to enable the left-hand side of the medical device 120 other portions of the medical device 120 to be submerged in a fluid, in some cases.

In a downward flush, such as that shown in FIG. 12, a fluid can travel from the right-hand side of the medical device 120 and exit out the left-hand side of the medical device 120. As the fluid travels down the medical device 120, air/particulates can also travel out the left-hand side of the medical device 120. In some cases, the fluid can pool/collect within the retention feature 204 and/or an engagement feature, such as the engagement feature 112(A). The tray 110, such as the engagement feature 112(A), can fill with the fluid. In some implementations, the left-hand side of the medical device 120 is submerged in a fluid at the left-hand side of the tray 110, which can enhance downward flushing.

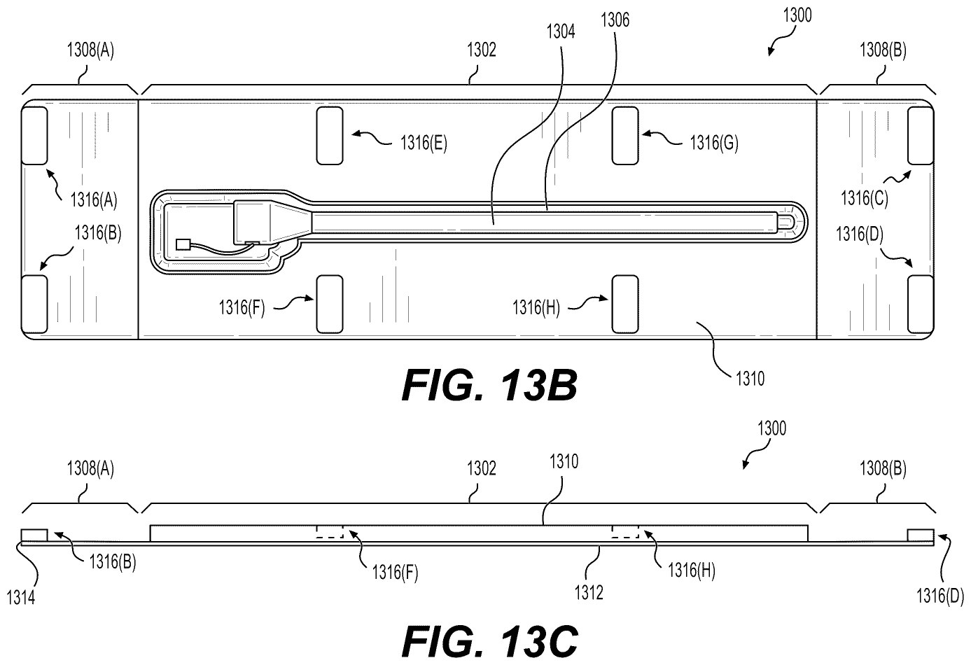

FIGS. 13A-13C illustrate example packaging/package/tray 1300 that includes one or more end portions that are configurable to cover/retain a medical instrument in accordance with one or more examples. In particular, FIG. 13A illustrates a perspective view of the packaging 1300, FIG. 13B illustrates a top view of the packaging 1300, and FIG. 13C illustrates a side view of the packaging 1300. Although various example features are shown in a particular configuration for the packaging 1300, other features can be implemented and/or the illustrated features can be configured in different manners. The packaging 1300 can be similar to or the same as (in various aspects) the packaging 100. In examples, the packaging 1300 is implemented as an integral component, while in other examples the packaging 1300 is implemented with several components/elements.

As shown, the packaging 1300 includes a retention/central section 1302 configured to retain/hold a medical instrument 1304. The retention section 1302 can include a retention feature 1306 that is configured to receive/hold/secure the medical instrument 1304 in a firm or loose manner within the packaging 1300. For example, the retention feature 1306 can include a recessed portion that extends along a length of the packaging 1300, as shown, which can be configured to hold the medical instrument 1304 in a fixed position within the packaging 1300 (e.g., the medical instrument is fully secured) or with a more loosely defined coupling to the packaging 1300 (e.g., the medical instrument is more loosely secured, such that the medical instrument 1304 can move somewhat within the recessed portion). Although the retention feature 1306 is illustrated as a recessed portion, the retention feature 1306 can be implemented in any number of ways, such as using one or more features to receive/hold one or more medical instruments. In the example shown, a single retention features is implemented; however, any number of retention features can be implemented to hold any number of medical devices. In some cases, the retention feature 1306 can be used as a reservoir to collect/contain a fluid, such as when a medical device is flushed with a fluid while being disposed within the packaging 1300.

The packaging 1300 can include one or more end portion/sections 1308 (also referred to as “the foldable/bendable/flexible portions 1308”) that are adjacent to and/or coupled to the retention section 1302. One or more of the end portions 1308 can be configured to flex/fold/bend relative to the retention section 1302. In some cases, one or more of the end portions 1308 are configured to fold/bend/flex using a scored portion (e.g., a formed groove in the material), a relatively thin piece of material (e.g., less than another portion of the packaging 1300, such as the central portion 1302), a hinge, or another feature that assists in providing a hinge/flexible point.

In a packaged configuration, such as that shown in FIGS. 14A and 14B, one or more of the end portions 1308 can fold upwards/around onto and/or couple/attach to a top side/surface 1310 of the packaging 1300 to cover/hold ends/end portions of the medical device 1304 disposed in the packaging 1300. In FIG. 14A, a section 1308(A)(1) is representative of sections where the end portions 1308(A), 1308(B) fold, wherein the enlargement/callout indicates that the end portion 1308(A) can fold upwards with or without one or more creases.

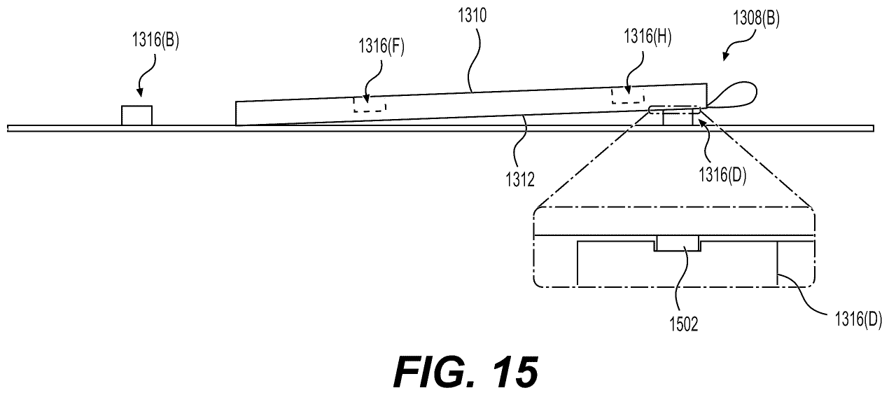

In a device-preparation configuration, such as that shown in FIG. 15, one or more of the end portions 1308 can fold back onto and/or couple/attach to a bottom side/surface 1312 of the packaging 1300 to elevate/raise one or more ends of the packaging 1300 and/or the medical device 1304 disposed in the packaging 1300. Here, the end portion 1308 can provide a support structure to elevate an end of the packaging 1300. In some implementations, the packaging 1300 includes an engagement feature 1502 on the bottom side/surface 1312, such as that shown in FIG. 15. Here, the engagement feature 1502 can be configured to engage/couple to an engagement feature 1316(D) to maintain/secure the end portion 1308(B) in a folded/bent configuration. The engagement feature 1502 can be the same as or similar to any of engagement features 1316 (discussed in further detail below). Further in some cases, the engagement feature 1316(D) can include one or more engagement features on multiple sides, such that the engagement feature 1316(D) can be configured to couple to the top side 1310 and the bottom side 1312 of the packaging 1300 at various times.

As shown, the packaging 1300 can include a base structure/sheet/portion 1314 (which can have an elongate form, in some cases) and/or one or more engagement/coupling/mating features 1316 disposed thereon. In some implementations, the base structure 1314 extends from one end of the packaging 1300 to the other end and provides flexibility for bending/flexing the one or more end portions 1308. That is, the end portions 1308 can form part of the base structure 1314 in some cases. In implementations, the base structure 1314 includes a sheet of packaging material coupled to the retention section 1302, wherein the retention section 1302 includes a thicker packaging section, such as with respect to the side view shown in FIG. 13C. For example, the packaging material can be dimensioned such that a depth of the packaging 1300 in the retention section 1302 can hold the medical device 1304 (e.g., be as thick as the medical device 1304). In FIGS. 13A-13C, the base structure 1314 (and the end portions 1308) are shown in a substantially straight/planar configuration, which can be a default configuration, in some cases.

One or more of the one or more engagement features 1316 can be configured to couple/attach to the top side 1310 of the packaging 1300, such as to secure the ends of the medical device 1304 in a packaged configuration, and/or to couple/attach to the bottom side 1312 to elevate one or more portions of the packaging 1300. For example, the engagement features 1316(A)-1316(D) can be configured as male engagement features configured to couple to/engage with the engagement feature 1316(E)-1316(H) implemented as female engagement features. Each engagement feature 1316 can be implemented in a variety of forms/manners to couple to another component/element, such as to facilitate a packaged configuration and/or a device-preparation configuration. For instance, an engagement feature 1316 can include one or more protrusions, detents, indentations, recesses, cavities, depressions, holes, projections, snap-fit features, pins, hooks, flanges, edges, nipples, fasteners, adhesives, etc. In examples, one or more of the engagement features 1316 can be the same as or similar to the engagement feature 112/132 discussed above.

For instance, in returning to the package configuration shown in FIGS. 14A and 14B, the male engagement feature 1316(A) can be configured to engage with the female engagement feature 1316(E) and the male engagement feature 1316(B) can be configured to engage with the female engagement feature 1316(F) when the end portion 1308(A) is folded onto the top surface 1310 of the packaging 1300. Similarly, the male engagement feature 1316(C) can be configured to engage with the female engagement feature 1316(G) and the male engagement feature 1316(D) can be configured to engage with the female engagement feature 1316(H) when the end portion 1308(B) is folded onto the top surface 1310 of the packaging 1300.

Although a particular number of engagement features 1316 are illustrated at specific locations with particular types of engagement features, the packaging 1300 can include any number of engagement features arranged in any location and/or implemented in a variety of manners.

As such, the packaging 1300 can be configurable for various functions, such as by using the end portions 1308 for covering/retaining/protecting the medical device 1304 in a packaged configuration and/or for propping-up the packaging 1300/medical device 1304 in a device-preparation configuration. In examples, the end portions 1308 can reduce the amount of packaging needed to retain/cover and/or elevate a medical instrument, in comparison to other solutions, such as retainer implementation where a retainer covers/spans a larger area of a tray/packaging. In implementations, the packaging 1300 (sometimes referred to as “the packaging 1300 with retention ends/end portions’) is implemented without a lid/cover/retainer (which is used in other solutions to retain a medical device). As such, the amount of packaging material can be minimized.

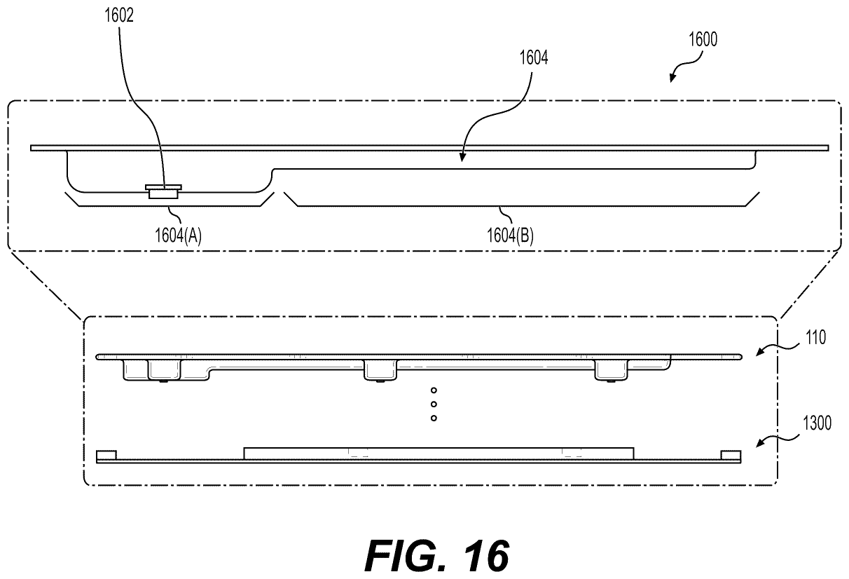

FIG. 16 illustrates example packaging/package 1600 that includes one or more plugs/stoppers/caps/sealing members 1602 (referred to as “the plug 1602” for convenience) that are configured to seal one or more holes in the packaging 1600 in accordance with one or more examples. The packaging 1600 can be similar to or the same as (in various aspects) the packaging 100 and/or the packaging 1300. The packaging 1300 can be representative/correspond to a tray (e.g., the tray 110), a retainer (e.g., the retainer 130), and/or another packaging component/element. As discussed herein, in various examples packaging is used to perform a device-preparation function, such as to flush or otherwise treat a medical device with a fluid. To assist the user in flushing or otherwise treating the medical device, the packaging 1600 (or any other packaging discussed herein) can include a feature (e.g., well, recess, tank, etc.) to collect the fluid and/or the plug 1602, which can be removably coupled to the feature at the appropriate times to seal the feature and/or drain fluid from the feature.

As shown in FIG. 16, the packaging 1600 includes one or more fluid containment features 1604, such as wells, recessed portions, receptacles, tanks, etc., configured to contain/hold/collect a fluid (not shown). The fluid containment feature 1604 can correspond to any of the engagement features, recessed portions, and/or other features discussed herein configured to collect a fluid. The fluid containment feature 1604 can include a first portion/section 1604(A) that is deeper (e.g., has more depth) than a second portion/section 1604(B). In implementations, the fluid containment feature 1604 corresponds to any of the recessed/retention portions discussed herein. For example, the first portion 1604(A) can correspond to the feature 112(A)(1) (e.g., recessed portion) for the tray 110 and/or the retention feature 1306 (e.g., recessed portion) of the packaging 1300. In some instances, the first portion 1604(A) provides a container to receive fluid when the packaging 1600 is elevated at one end, such as elevated at the right-hand side of the packaging 1600 with respect to FIG. 16. As also shown, the first portion 1604(A) can include the plug 1602, which can be removably attached in a bottom of the first portion 1604(A). In the example shown, the plug 1602 is sealed within a hole/opening/aperture/space/orifice in the first portion 1604(A). However, the plug 1602 can be removed, as discussed below.

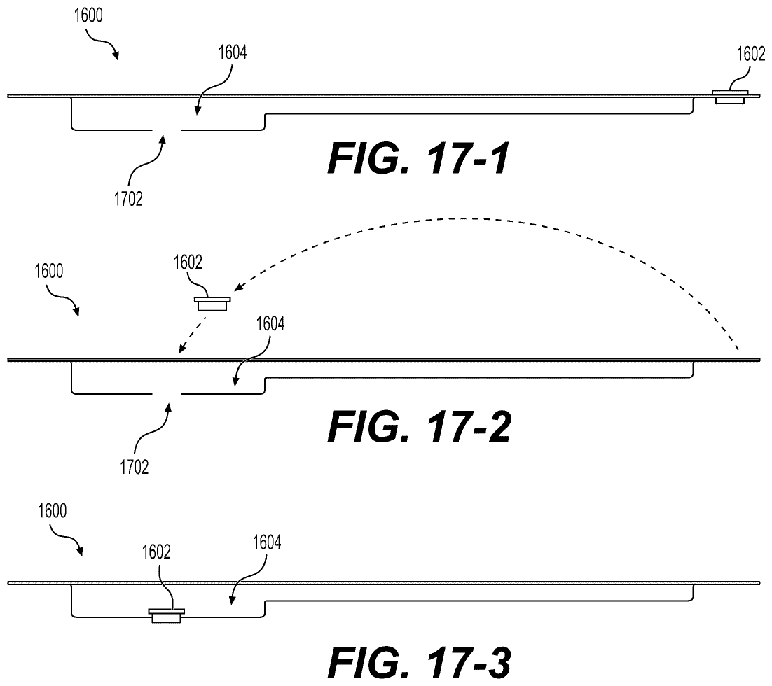

The plug 1602 can be implemented in a variety of manners, such as with a portion of the packaging 1600, fastener, adhesive, etc. For example, the plug 1602 can be part of the packaging 1600 and configured to be removed (e.g., part of a tray, retainer, etc.), a separate component, etc. In implementations, the plug 1602 is pre-seated in the hole in the fluid containment feature 1604, such that the packaging 1600 is ready to receive/collect a fluid when the rest of the packaging 1600 is opened (e.g., removed from any external packaging). Further, in implementations, as shown in FIGS. 17-1 through 17-3, the plug 1602 is initially, such as during distribution/shipment, located at a first location within the packaging 1600 that is a distance from a hole/opening 1702 in which plug 1602 will be implemented (as shown in FIG. 17-1). The plug 1602 is then removed from the first location (as shown in FIG. 17-2), such as by a user or device, and placed/disposed in the hole 1702 (as shown in FIG. 17-3). The plug 1602 can be configured to seal the fluid containment feature 1604 to prevent a fluid from leaking out of the fluid containment feature 1604. In such configuration, the packaging 1600 is configured for a device-preparation operation/function, such as flushing of a medical instrument, which can be disposed within the packaging 1600. Moreover, in implementations, the plug 1602 is located adjacent to the hole 1702 and flipped over (e.g., bend/flex) into the hole 1702 when desired. Further, in implementations, the plug 1602 is located in another packaging component, such as a retainer or another element that is associated with the packaging 1600.

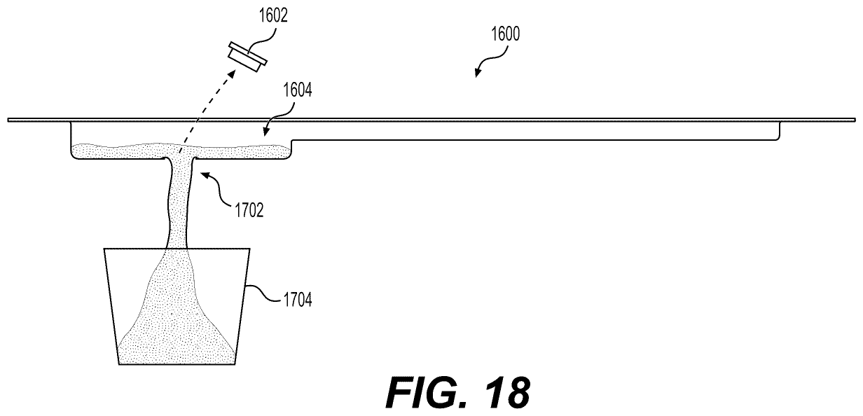

As shown in FIG. 18, fluid 1702 can collect in the fluid containment feature 1604 of the packaging 1600, such as when a medical device (not shown) is being flushed while located in the packaging 1600. When desired, such as upon flushing the medical device with the fluid 1702, the plug 1602 can be removed to allow the fluid 1702 to drain out into a container/vessel 1704 for disposal of the fluid 1702. This can allow the fluid 1702 to be disposed of without spilling the fluid 1702.

In implementations, a manufacturing process can be implemented to form/create any of the packaging/packaging features discussed herein.

Any example methods and/or structures disclosed herein for treating a patient (e.g., performing a procedure) can also encompass analogous methods and/or structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

### Additional Description of Examples

Provided below is a list of examples, each of which may include aspects of any of the other examples disclosed herein. Furthermore, aspects of any example described above may be implemented in any of the numbered examples provided below.

Example 1: A packaging system comprising: a tray including: a retention section that includes a retention feature on a first side of the tray, the retention feature being configured to retain a medical device; and a first end portion coupled to the retention section and configured to bend relative to the retention section; wherein the packaging system is configurable to: (i) a first configuration in which the first end portion is at least one of substantially straight relative to a longitudinal axis of the tray or bent towards the first side of the tray; and (ii) a second configuration in which the first end portion is bent towards a second side of the tray to provide a support structure for one end of the tray.

Example 2: The packaging system of any example herein, in particular example 1, wherein the first end portion of the tray is configured to bend at multiple locations to form multiple sections that incrementally bend towards the second side of the tray.

Example 3: The packaging system of any example herein, in particular example 2, wherein at least one of the multiple sections is configured to bend to contact the second side of the tray.

Example 4: The packaging system of any example herein, in particular examples 1-3, wherein, in the second configuration, the first end portion of the tray is configured to couple to the second side of the tray.

Example 5: The packaging system of any example herein, in particular example 1-4, further comprising: a retainer removably attachable to the tray; wherein, in the first configuration, the retainer is coupled to the first side of the tray to retain the medical device in the retention feature, and, in the second configuration, the retainer is positioned adjacent to the second side of the tray to receive the first end portion of the tray.

Example 6: The packaging system of any example herein, in particular example 5, wherein the tray includes a first engagement feature disposed on the second side of the tray and the retainer includes a second engagement feature configured to couple to the first engagement feature when the retainer is configured in the second configuration.

Example 7: The packaging system of any example herein, in particular examples 1-6, wherein, in the first configuration, the first end portion is bent onto the first side of the tray to cover at least a portion of the medical device.

Example 8: The packaging system of any example herein, in particular example 7, wherein the tray includes a first engagement feature disposed on the first side of the tray and the first end portion includes a second engagement feature, and, in the first configuration, the first engagement feature is coupled to the second engagement feature.

Example 9: The packaging system of any example herein, in particular example 7, wherein the tray further includes a second end portion, and, in the first configuration, the second end portion is bent onto the first side of the tray to cover at least another portion of the medical device.

Example 10: The packaging system of any example herein, in particular examples 1-9, further comprising: a plug removably attachable to a hole in a fluid containment feature within the tray.

Example 11: The packaging system of any example herein, in particular example 10, wherein the plug is located at a position in the tray a distance from the hole and configured to be removed from the position.

Example 12: The packaging system of any example herein, in particular examples 1-11, wherein at least one of the packaging system or the medical device is sterilized.

Example 13: A packaging system comprising: a tray including a retention feature on a first side of the tray, the retention feature being configured to retain a medical device; and a retainer removably attachable to the tray; wherein the packaging system is configurable to: (i) a first configuration in which the retainer is coupled to the first side of the tray to retain the medical device in the retention feature; and (ii) a second configuration in which the retainer is coupled to a second side of the tray to elevate a first end of the tray.

Example 14: The packaging system of any example herein, in particular example 13, wherein the tray includes a first engagement feature on the first side of the tray and the retainer includes a second engagement feature configured to couple to the first engagement feature when the packaging system is configured in the first configuration.

Example 15: The packaging system of any example herein, in particular example 13 or claim 14, wherein the tray includes a first engagement feature on the second side of the tray and the retainer includes a second engagement feature configured to couple to the first engagement feature when the packaging system is configured in the second configuration.

Example 16: The packaging system of any example herein, in particular examples 13-15, wherein, in the first configuration, the retainer is positioned in a first orientation with a first side of the retainer coupled to the first side of the tray, and, in the second configuration, the retainer is positioned in a second orientation with the first side of the retainer coupled to the second side of the tray.

Example 17: The packaging system of any example herein, in particular examples 13-16, wherein the retainer includes an engagement feature that is configured to couple to the first side of the tray when the packaging system is configured in the first configuration and couple to the second side of the tray when the packaging system is configured in the second configuration.

Example 18: The packaging system of any example herein, in particular examples 13-17, wherein the retainer is configured to flex towards a surface of the retainer to implement a folded configuration and the retainer is configured to, in the second configuration, couple to the tray in the folded configuration.

Example 19: The packaging system of any example herein, in particular examples 13-18, wherein the retainer is configured to separate into multiple pieces and couple to each other and the retainer is configured to, in the second configuration, couple to the tray when the multiple pieces of the retainer are coupled together.

Example 20: The packaging system example herein, in particular examples 13-19, further comprising: a plug removably attachable to a hole in a fluid containment feature within the tray.

Example 21: The packaging system of any example herein, in particular example 20, wherein the plug is located at a position in the tray a distance from the hole and configured to be removed from the position.

Example 22: The packaging system of any example herein, in particular example 20, wherein the plug is located in the retainer and configured to be removed from the retainer.

Example 23: The packaging system of any example herein, in particular examples 13-22, wherein at least one of the packaging system or the medical device is sterilized.

Example 24: Packaging comprising: a tray including a retention section configured to retain a medical device and a first end portion coupled to the retention section, the tray being configurable to: (1) a first state in which the first end portion is flexed towards and covers at least a portion of a first side of the tray; and (ii) a second state in which the first end portion is flexed towards a second side of the tray to provide a support structure.

Example 25: The packaging of any example herein, in particular example 24, wherein the first end portion of the tray is configured to bend at multiple locations to form multiple sections that incrementally bend towards the second side of the tray in the second state.

Example 26: The packaging of any example herein, in particular example 25, wherein at least one of the multiple sections is configured to bend to contact the second side of the tray.

Example 27: The packaging of any example herein, in particular examples 24-26, wherein, in the second state, the first end portion of the tray is configured to couple to the second side of the tray.

Example 28: The packaging of any example herein, in particular examples 24-27, further comprising: a retainer configured to, in the first state, attach to the first side of the tray.

Example 29: The packaging of any example herein, in particular examples 24-28, wherein the tray includes a first engagement feature disposed on the first side of the tray and the first end portion includes a second engagement feature, and, in the first state, the first engagement feature is coupled to the second engagement feature.

Example 30: The packaging of any example herein, in particular example 29, wherein the tray further includes a second end portion, and, in the first state, the second end portion is flexed towards and covers at least another portion of the first side of the tray.

Example 31: The packaging of any example herein, in particular examples 24-30, further comprising: a plug removably attachable to a hole in a fluid containment feature within the tray.

Example 32: The packaging of any example herein, in particular example 31, wherein the plug is located at a position in the tray a distance from the hole and configured to be removed from the position.

Example 33: The packaging of any example herein, in particular examples 24-32, wherein at least one of the packaging or the medical device is sterilized.

Example 34: Packaging comprising: a tray configured to retain a medical device; and a retainer removably attachable to the tray; wherein the packaging is configurable to: (i) a first state in which the retainer is attached to a first side of the tray; and (ii) a second state in which the retainer is attached to a second side of the tray to elevate a first end of the tray.

Example 35: The packaging of any example herein, in particular example 34, wherein the tray includes a first engagement feature on the first side of the tray and the retainer includes a second engagement feature configured to couple to the first engagement feature when the packaging system is configured in the first state.

Example 36: The packaging of any example herein, in particular example 34 or claim 35, wherein the tray includes a first engagement feature on the second side of the tray and the retainer includes a second engagement feature configured to couple to the first engagement feature when the packaging system is configured in the second state.

Example 37: The packaging of any example herein, in particular examples 34-36, wherein the retainer includes an engagement feature that is configured to couple to the first side of the tray when the packaging is configured in the first state and couple to the second side of the tray when the packaging is configured in the second state.

Example 38: The packaging of any example herein, in particular examples 34-37, wherein the retainer is configured to fold to a folded configuration and the retainer is configured to, in the second state, couple to the tray in the folded configuration.

Example 39: The packaging of any example herein, in particular examples 34-38, wherein the retainer is configured to separate into multiple pieces and couple to each other and the retainer is configured to, in the second state, couple to the tray when the multiple pieces of the retainer are coupled together.

Example 40: The packaging of any example herein, in particular examples 34-39, further comprising: a plug removably attachable to a hole in a fluid containment feature within the tray.

Example 41: The packaging of any example herein, in particular example 40, wherein the plug is located at a position in the tray a distance from the hole and configured to be removed from the position.

Example 42: The packaging of any example herein, in particular example 40, wherein the plug is located adjacent to the hole.

Example 43: The packaging of any example herein, in particular examples 34-42, wherein the first state is a packaged state and the second state is a device-preparation state in which the medical device is elevated within the packaging at one end.

Example 44: The packaging of any example herein, in particular examples 34-43, wherein the tray includes a recessed portion configured to retain the medical device and contain a fluid.

Example 45: The packaging of any example herein, in particular examples 34-44, further comprising: the medical device.

Example 46: The packaging of any example herein, in particular example 45, wherein the medical device includes a tube configured to receive a fluid.

Example 47: The packaging of any example herein, in particular examples 34-46, wherein, in the first state, the retainer is positioned in a first orientation with a first side of the retainer coupled to the first side of the tray, and, in the second state, the retainer is positioned in a second orientation with the first side of the retainer coupled to the second side of the tray.

Example 48: The packaging of any example herein, in particular examples 34-47, wherein the tray includes a retention feature configured to secure the medical device to the tray.

Example 49: The packaging of any example herein, in particular examples 34-48, wherein the packaging is sterilized.

Example 50: The packaging of any example herein, in particular examples 34-49, wherein the medical device is sterilized.

Depending on the example, certain acts, events, or functions of any of the processes or algorithms described herein can be performed in a different sequence, can be added, merged, and/or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, can encompass the concepts of equality. For example, “less” can mean not only “less” in the strictest mathematical sense, but also “less than or equal to.”

What is claimed is:

1. A packaging system comprising:
a tray including:
a retention section that includes a retention feature on a first side of the tray, the retention feature being adapted to retain a medical device; and
a first end portion coupled to the retention section and adapted to bend relative to the retention section;
wherein the packaging system is adaptable to:
(i) a first state in which the first end portion is bent towards the first side of the tray and coupled to a first engagement feature on the first side of the tray to hold the medical device; and
(ii) a second state in which the first end portion is bent towards a second side of the tray and coupled to a second engagement feature on the second side of the tray to provide a support structure for one end of the tray.

2. The packaging system of claim 1, wherein the tray includes a sheet of material that includes the retention section and the first end portion.

3. The packaging system of claim 1, wherein the first end portion of the tray is adapted to bend at multiple locations to form multiple sections that incrementally bend towards the second side of the tray.

4. The packaging system of claim 3, wherein at least one of the multiple sections is adapted to bend to contact the second side of the tray.

5. The packaging system of claim 1, further comprising:
a retainer removably attachable to the tray;
wherein, in the first state, the retainer is coupled to the first side of the tray to retain the medical device in the retention feature, and, in the second state, the retainer is positioned adjacent to the second side of the tray to receive the first end portion of the tray.

6. The packaging system of claim 1, wherein the tray further includes a second end portion, and, in the first state, the second end portion is bent onto the first side of the tray to cover the medical device.

7. The packaging system of claim 1, further comprising:
a plug removably attachable to a hole in a fluid containment feature within the tray, the plug is located at a position in the tray a distance from the hole and adapted to be removed from the position.

8. A packaging tray comprising:
a retention section that includes a retention feature on a first side of the packaging tray, the retention feature being adapted to retain a medical device; and
a first end portion coupled to the retention section and adapted to bend relative to the retention section, the first end portion being adaptable to:
(i) a first configuration in which the first end portion is bent towards the first side of the packaging tray and coupled to a first engagement feature on the first side of the packaging tray; and
(ii) a second configuration in which the first end portion is bent towards a second side of the packaging tray and coupled to a second engagement feature on the second side of the packaging tray to provide a support structure for one end of the packaging tray.

9. The packaging tray of claim 8, wherein the retention section and the first end portion are formed of a same sheet of material.

10. The packaging tray of claim 8, wherein the first end portion of the packaging tray is adapted to bend at multiple locations to form multiple sections that incrementally bend towards the second side of the packaging tray.

11. The packaging tray of claim 10, wherein at least one of the multiple sections is adapted to bend to contact the second side of the packaging tray.

12. The packaging tray of claim 8, wherein the packaging tray further includes a second end portion, and, in the first configuration, the second end portion is bent onto the first side of the packaging tray to cover the medical device.

13. The packaging tray of claim 8, wherein at least one of the packaging tray or the medical device is sterilized.

14. A packaging tray comprising:
a retention section that includes a retention feature on a top side of the packaging tray to receive a medical device; and
a flexible section adapted to flex relative to the retention section, the flexible section to implement:
(i) a packaged configuration in which the flexible section flexes in a first direction and attaches to the top side of the packaging tray; and
(ii) a device-preparation configuration in which the flexible section flexes in a second direction and attaches to a bottom side of the packaging tray.

15. The packaging tray of claim 14, wherein the retention section and the flexible section are formed of a same sheet of material.

16. The packaging tray of claim 14, wherein the flexible section is adapted to bend at multiple locations to form multiple sections that incrementally bend towards the bottom side of the packaging tray.

17. The packaging tray of claim 14, wherein the packaging tray further includes another flexible section, and, in the packaged configuration, the other flexible section flexes and attaches to the top side of the packaging tray.

18. The packaging tray of claim 14, further comprising:
a retainer removably attachable to the packaging tray;
wherein, in the packaged configuration, the retainer is coupled to the top side of the packaging tray to retain the medical device in the retention feature, and, in the device-preparation configuration, the retainer is positioned adjacent to the bottom side of the packaging tray to receive the flexible section.

19. The packaging tray of claim 14, further comprising:
a plug removably attachable to a hole in a fluid containment feature within the packaging tray, the plug is located at a position in the packaging tray a distance from the hole and adapted to be removed from the position.

20. The packaging tray of claim 19, wherein the flexible section is located on one side of the packaging tray and the position of the plug is located on another side of the packaging tray.
